(19) United States
(12) Patent Application Publication
(10) Pub. No.: US 2021/0254459 A1
(43) Pub. Date: Aug. 19, 2021

(54) IN-LINE MUD LOGGING SYSTEM

(71) Applicant: SHELL OIL COMPANY, HOUSTON, TX (US)

(72) Inventors: Anton NIKITIN, Houston, TX (US); Amie Marie HOWS, Houston, TX (US); Ronny HOFMANN, Houston, TX (US)

(21) Appl. No.: 17/252,631

(22) PCT Filed: Jun. 18, 2019
(86) PCT No.: PCT/US2019/037647

§ 371 Date: Dec. 15, 2020

Related U.S. Application Data
(60) Provisional application No. 62/687,451, filed on Jun. 20, 2018.

(51) Int. Cl. E21B 49/00; G01N 23/12; G01N 33/28; G01V 5/06

(52) U.S. Cl. CPC E21B 49/005; G01N 23/12; G01N 2223/043; G01V 5/06; G01N 33/2823

(57) ABSTRACT

A system for real-time or near real-time analysis of drilling fluid, drill cuttings and/or mud gas transported from a well to a mud circulation system has a hydrocarbon analysis device for in-line measuring hydrocarbon content in the material and an in-line gamma-ray detection apparatus. The in-line gamma-ray detection apparatus is disposed in the path of material transported from the well. A control unit for real-time or near real-time receives and correlates signals from the hydrocarbon analysis device and the in-line gamma-ray detection apparatus.

### CROSS REFERENCE TO RELATED APPLICATIONS

This application claims the benefit of U.S. Provisional Application No. 62/687,451, filed 20 Jun. 2018, which is incorporated herein by reference.

### FIELD OF THE INVENTION

The present invention relates to mud logging and, in particular, to an in-line mud logging system for improved sampling and real- or near real-time analysis of drill cuttings.

### BACKGROUND OF THE INVENTION

In drilling operations for extracting hydrocarbons from a subsurface formation, drilling mud and/or drilling fluid (hereinafter “drilling fluid” will refer to drilling mud and/or other drilling fluids) is typically pumped downhole to carry drill cuttings to the surface. The drill cuttings, circulating drilling fluid and mud gas can provide useful information regarding strata in the subsurface formation, including, for example, without limitation, lithological type of a formation, the quality of the rock, mapping of a formation, likelihood of missed or new hydrocarbon layers or reservoirs, and the like.

Mud logging is one of the oldest methods for collecting subsurface information. Conventionally, drill cuttings carried to the surface are separated from the circulation fluid and fines with a shaker.

Typically, so-called mud loggers are on-site at the drilling operation for manually collecting a sample of drill cuttings from a conveyor, such as a shaker. In a conventional operation, a catcher board is placed at the base of the conveyor to catch drill cuttings as they fall from the end of the conveyor. After the desired sampling time, the mud logger scrapes the cuttings off the catcher board into a sample container and labels the samples for later analysis, off-site or on-site at a location remote from the conveyor.

Concentrations of naturally occurring radioactive isotopes such as K, U and Th are widely used in formation evaluation. Currently these parameters of formation are measured downhole by gamma-ray wireline tools, for example Natural Gamma-ray Spectroscopy (NGS) wireline tools, and/or measured on cuttings or core samples in the lab using X-ray Fluorescent Spectroscopy (XRF). In the case of wireline tools, substantial additional costs are imposed due to rig time required for this operation. For XRF measurements, there are limitations on the number of sample measurements a mud logger can physically process and characterize on-site.

If information delivered by mud logging is intended for the quantitative description of the subsurface, drill cuttings samples should be acquired every 5 feet (1.5 m) along a well borehole. At a typical rate of penetration (ROP) of 100 ft/hr (30 m/hr), a sample of drill cuttings material would need to be acquired every 3 minutes. However, this rate of drilling cutting sample acquisition is not always feasible. Beyond the physical demands of a sample acquisition every 3 minutes, on-site analysis becomes challenging due to a limited throughput of lab XRF equipment. Furthermore, there are safety concerns for people performing such sample catching operation. Accordingly, it would be advantageous to reduce or eliminate the need for a person taking manual samples, while improving a sample acquisition rate.

In an effort to address the need for drill cutting sampling and analysis of sampled cuttings material, U.S. Pat. No. 6,386,026 B1 (Zamfes) describes analyzing a continuous stream of drill cuttings by conveying the cuttings from a shaker to a trough having a helical screw for moving the cuttings. A metering device holds a sample between vanes and releases one portion of the sample to a cotton bag and passes another portion to another screw conveyor to a transparent cylinder for quantitative analysis. In another approach, Zamfes (US2005/0082468A1, US2008/0202811A1) contemplates modifying the device of U.S. Pat. No. 6,386,026B1 with gamma radiation, beta radiation and sonic sensors provided on the side of an auger transporting drill cuttings. It is unclear from these publications, but it appears that Zamfes intended the auger with sensors would replace the transparent cylinder described in U.S. Pat. No. 6,2386,026B1.

U.S. 7,730,795B2 (Rieberer) relates to a drill cuttings sampler having a frame for mounting on the end of a conventional vibratory shaker for supporting a perforated plate in the outflow path of drill cuttings exiting the shaker. A removable cutting collector is mounted beneath the perforated plate for allowing cuttings of a desired size to pass through to the collector, while larger cuttings are rejected and pass over the plate by washing with spray nozzles and vibration from the vibratory shaker. Samples of a pre-determined drilling interval are removed from the cutting collector for remote analysis.

EP3156587A1 (Schlumberger) describes an automated device for analyzing drill cuttings. A sampler located at the end of a shaker has at least one conveyor for transporting a sample in a canister from an outlet of a shaker to a treatment unit. A robotic manipulator takes the canister from the conveyor to a washing station and an archiving station for tagging and storing the sample.

WO2012/100283A1 (Technological Resources Pty) relates to a sampling and analysis system for coupling with a drilling rig. A frame supports a drill cuttings handling system and an elemental analysis device and is coupled to a drilling rig platform with hydraulic rams. Drill cuttings are transported from a conveyor near the hole being drilled to a sampler splitter to produce two or more sample streams. One stream is directed to a feed chute and then to another conveyor in the analyzer, through a levelling device. Once this portion of the sample is transported past the analysis zone of the analyzer, it falls off the downstream end to the ground. The analyzer and conveyor and related equipment, are supported on a vibration isolation system.

Deficiencies of the systems described above include not allowing for real-time or near real-time analysis useful for more efficient decision-making, requiring modifications to drilling operation equipment, and/or they are very complex. Still another deficiency of the prior system having sensors on a transport tube is that the drill cuttings are not concentrated enough to measure a meaningful result. While it may be suitable for some applications, diluted drill cutting samples are a problem when deeper wells are drilled, where significantly more drilling fluid is required to remove the drill cuttings from deeper depths. There is a need for a simple system that can be implemented in an existing drilling operation without undue modification. There is also a need for an in-line system that can concentrate drill cuttings for a more meaningful result.

### SUMMARY OF THE INVENTION

According to one aspect of the present invention, there is provided an in-line mud logging system for real-time or near real-time analysis of material selected from drilling fluid, drill cuttings, mud gas and combinations thereof from a well, the material having at least a solids content and a liquid content, the material transported from the well to a mud circulation system, the mud circulation system comprising a mud pipeline, a conveyor and a possum belly, the conveyor having a proximate end in material communication with the possum belly and a distal end from which the material falls from the conveyor, the in-line mud logging system comprising: a hydrocarbon analysis device for in-line measuring hydrocarbon content in the material; an in-line gamma-ray detection apparatus, the in-line gamma-ray detection apparatus disposed in the path of material transported from the well; and a control unit for real-time or near real-time receiving and correlating signals from the hydrocarbon analysis device and the in-line gamma-ray detection apparatus.

### BRIEF DESCRIPTION OF THE DRAWINGS

The system of the present invention will be better understood by referring to the following detailed description of preferred embodiments and the drawings referenced therein, in which:

FIG. 1 is a side elevation view illustrating the flow of material from a well to one embodiment of an in-line mud logging system of the present invention for real-time or near real-time analysis of material from the well (for better showing particular features of the invention, the drawing is not necessarily to scale);

FIG. 2 is a side elevation view illustrating the flow of material from a well to another embodiment of an in-line mud logging system of the present invention for real-time or near real-time analysis of material from the well (for better showing particular features of the invention, the drawing is not necessarily to scale);

FIG. 3 is a front perspective view of one embodiment of an in-line gamma-ray detection apparatus suitable for use in the system of the present invention, particularly the embodiment of FIG. 1;

FIG. 4 is a front elevation view of the apparatus of FIG. 3;

FIG. 5 is a top plan view of the apparatus of FIG. 3;

FIG. 6 is a front perspective view of the analysis body of the apparatus of FIG. 3 showing a sample accumulation tray in an open position;

FIG. 7 is a cross-sectional view of an analysis body of the apparatus of FIG. 3 along the line 7-7;

FIG. 8 is a cross-sectional view of another embodiment of an in-line gamma-ray detection apparatus suitable for use in the system of the present invention;

FIGS. 9A and 9B are cross-sectional views of the in-line gamma-ray detection apparatus of FIG. 8, showing operation of the in-line gamma-ray detection apparatus;

FIG. 10 is a cross-sectional view of one embodiment of an in-line hydrocarbon analysis device of the present invention suitable for use in the system of the present invention; and

FIG. 11 illustrates one embodiment of an interpretation scheme generated by the system of the present invention to convert acquired data into sub-surface information.

### DETAILED DESCRIPTION OF THE INVENTION

The present invention provides an in-line mud logging system for real-time or near real-time analysis of material from a well. The system has an in-line hydrocarbon analysis device, an in-line gamma-ray detection apparatus and a control unit for real-time or near real-time receiving and correlating signals from the hydrocarbon analysis device and the gamma-ray detection apparatus. The material transported from a well has at least a solids content and a liquid content. The material may also have a gas content. Material from the well is selected from drilling fluid, drill cuttings, mud gas and combinations thereof. The in-line mud logging system of the present invention reduces the need for mud-logging personnel to manually collect samples, for example, from a conventional catcher board. Moreover, samples can be taken at a higher frequency, thereby providing greater vertical resolution of a well.

Because drill cuttings comprise pieces of the formation crushed by the drill bit during a drilling operation, properties of these cuttings are representative of the properties of the formation from which the cuttings originated. As such, if traced back to a wellbore depth, these cuttings may be sampled and analyzed to provide information about the formation properties present at that depth within the wellbore.

The formation properties determined by the system of the present invention provide information to assess the ability of a formation to produce hydrocarbons and/or to estimate the mechanical properties of the formation, for example, to determine frackability of the formation.

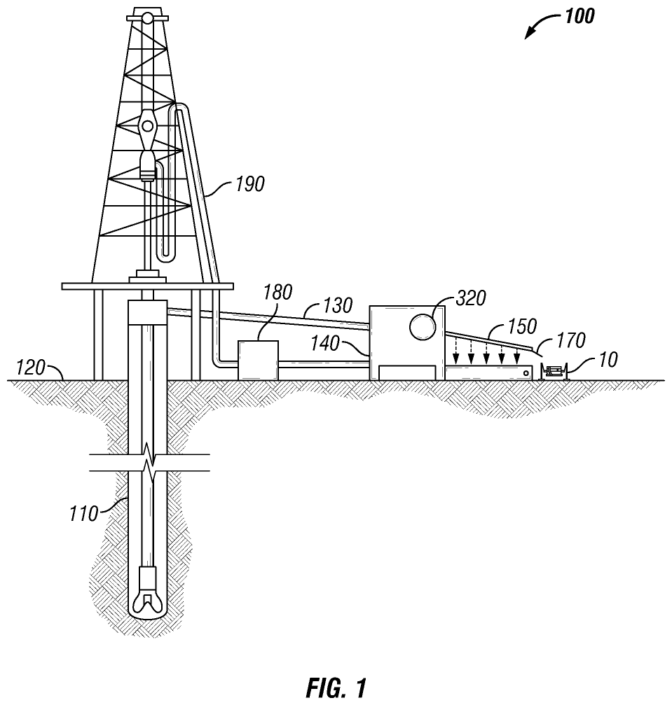

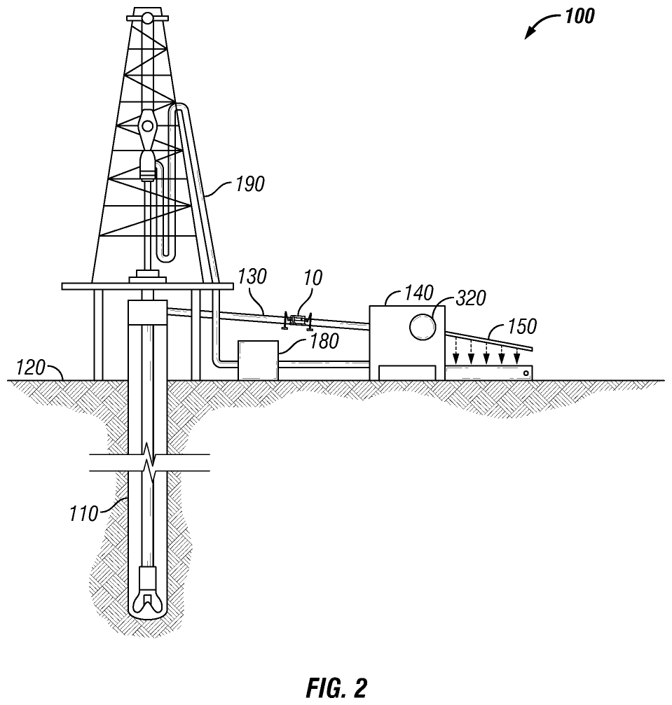

Two embodiments of a mud circulation system 100 of the present invention are depicted in FIGS. 1 and 2. Drill cuttings are transported from a well 110 to the surface 120 with drilling fluid. A mud pipeline 130 conveys material from the well 110 to a possum belly 140 which allows any entrained gasses to be released. The possum belly 140 also reduces the mud flow rate before the drilling fluid is transported to a conveyor 150. The conveyor 150 may be a shaker conveyor, a vibratory conveyor or a combination thereof. A proximate end of the conveyor 150 receives material, via the mud pipeline 130 and the possum belly 140, from the well 110. Preferably, the conveyor 150 acts to separate a majority of the drilling fluid from the drill cuttings, as depicted by the downward arrows from the conveyor 150. The drill cuttings are separated from the drilling fluid and the drilling fluid is returned downhole, with or without further treatment, to transport more drill cuttings to the surface 120.

In the embodiments of FIGS. 1 and 2, the drilling fluid is returned to the well 110 using a mud pump 180 and tubing 190. Drilling fluid can be exposed to the other treatments after being cleaned at the conveyor 150 before returning to the well bore, for example, without limitation, treatment in a settling tank and the like.

An in-line gamma-ray detection apparatus 10 is disposed in the path of material transported from the well. In the embodiment of FIG. 1, the gamma-ray detection apparatus 10 is disposed at the distal end of the conveyor 150. In the embodiment of FIG. 2, the gamma-ray detection apparatus 10 is disposed at the mud pipeline 130.

In the embodiment shown in FIG. 1, material falls from a distal end of the conveyor 150 onto the in-line gamma-ray detection apparatus 10. In a preferred embodiment of the present invention, the in-line gamma-ray detection apparatus 10 is deployed at the distal end of the conveyor 150 in a location typically reserved for a conventional catcher board, so that little to no retrofit of the mud circulation system 100 is required.

Optionally, as shown in the embodiment of FIG. 1, a funnel 170 is provided on or near the distal end of the conveyor 150 for selectively channeling all or a portion of the material from the conveyor 150 to the gamma-ray detection apparatus 10.

In the embodiment shown in FIG. 2, the gamma-ray detection apparatus 10 is placed at the mud pipeline 130.

In a preferred embodiment of the embodiment of FIG. 1, the in-line mud logging system 100 of the present invention uses an embodiment of an apparatus described in co-pending application entitled “In-line Mud Logging Apparatus” filed in the USPTO on the same day as the present application, as a provisional application, the entirety of which is incorporated by reference herein.

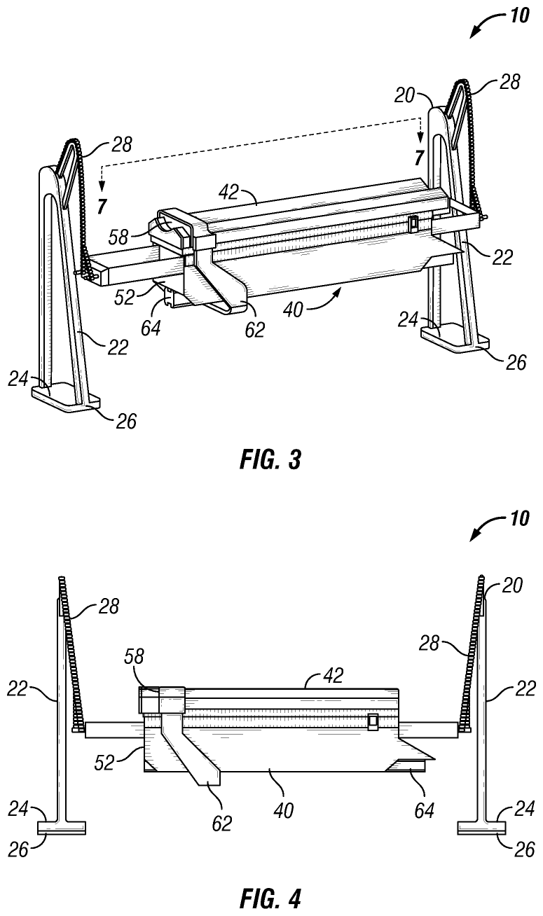

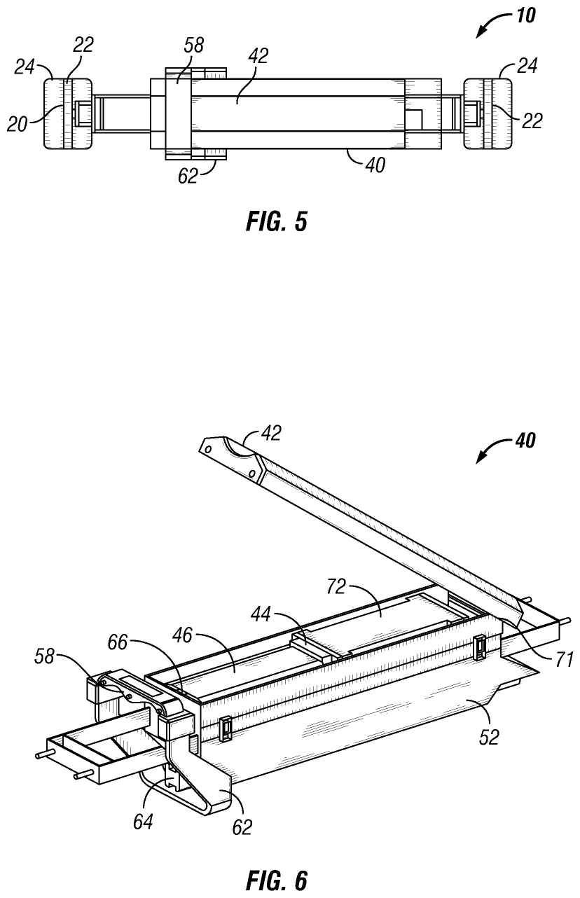

A particularly preferred embodiment of the in-line gamma-ray detection apparatus 10 for the system embodiment of FIG. 1 is depicted in FIGS. 3-7. An in-line gamma-ray detection apparatus 10 has a mounting frame 20 and an analysis body 40. The in-line gamma-ray detection apparatus 10 is adapted to be in material communication with the conveyor 150, for example, in a conventional mud circulation system. By “material communication,” we mean that the gamma-ray detection apparatus 10 receives material, for example, in the form of drill cuttings, directly from a conveyor. Preferably, the gamma-ray detection apparatus 10 receives material from the conveyor 150 without being directly attached to the conveyor 150. As mentioned above, an optional funnel 170 (shown in FIG. 1) may be used to facilitate material communication.

In the FIG. 1 embodiment, a preferred embodiment of an in-line mud gamma-ray apparatus 10 is provided with the mounting frame 20 to vibrationally isolate the analysis body 40 from vibrations in the mud circulation system 100 and surrounding equipment. As shown in FIG. 3, in one embodiment, the mounting frame 20 is a two-part structure with two supporting legs 22 that are not directly connected to one another. Alternatively, the supporting legs 22 are connected to one another with crossbar (not shown). The supporting legs 22 have a base 24 for supporting the analysis body 40 on the ground, a platform or other surface. Preferably, the base 24 is provided with a vibration-damping material 26. A particularly suitable vibration-damping material 26 is a rubber material, for example, natural, synthetic or recycled rubber, a rubber composite or a rubber laminate. However, other materials for suitable for vibration-damping will be apparent to those skilled in the art. Preferably, the vibration-damping material 26 can suppress or damp higher frequency vibrations.

In a preferred embodiment, the analysis body 40 is suspended from the mounting frame 20 with chains 28. The chains 28 advantageously suppress or damp lower frequency vibrations.

The analysis body 40 has a sample accumulation tray 42 to collect material falling from the conveyor. Because the cuttings are wet, at least a portion of the material tends to fall directly onto the sample accumulation tray 42 as opposed to falling forward of the sample accumulation tray 42. It has been observed that the material falling from the conveyor 150 will fall onto any material in the sample accumulation tray 42 until a substantially steady state is reached where any new material rolls off the material already in place or displaces some material previously accumulated in the sample accumulation tray 42. Accordingly, the sample obtained is generally a representative sample. Moreover, the sample is a concentrated sampling of a drilling interval, allowing for more meaningful results.

In the embodiment shown in FIGS. 3-7, the sample accumulation tray 42 acts as a cover for the interior of the analysis body 40, which houses a gamma-ray detector 44, preferably a gamma-ray spectrometer.

Natural Gamma-ray Spectroscopy (NGS) analysis of drill cuttings provides information about the type of rock being drilled. Such information is useful for drilling decisions. Potassium (K), thorium (Th), and uranium (U) are three natural sources of gamma-ray radiation present in the earth. Each of these elements emit gamma-rays with known energies, which are unique to the particular element. Shales can be distinguished from other types of rock due to the relatively high levels of these gamma-ray radiating elements present in shale. Accordingly, the presence of shale is useful information for making decisions about a drilling operation.

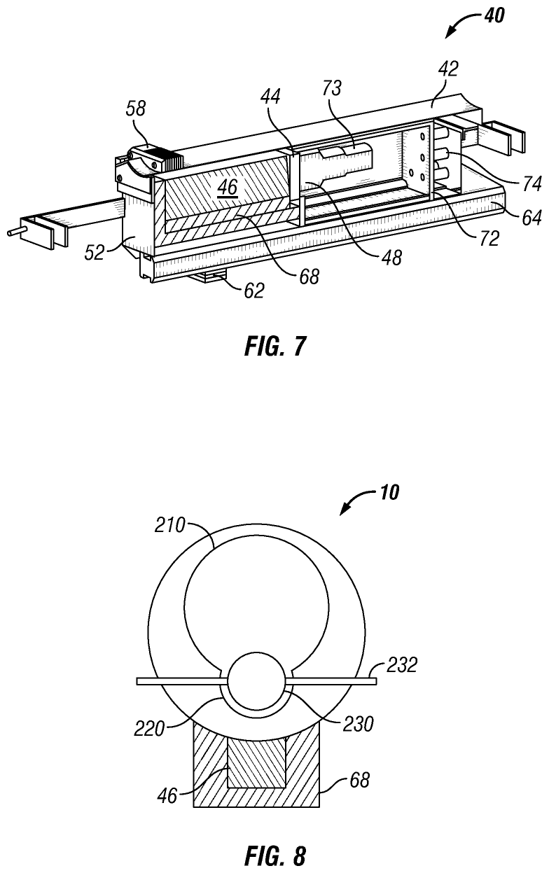

FIG. 6 illustrates the sample accumulation tray 42 in an open position to reveal the gamma-ray detector 44, while FIG. 7 is a cross-sectional view of the analysis body 40. The gamma-ray detector 44 preferably comprises a gamma-ray detecting scintillation crystal 46, which is preferably optically coupled to a photomultiplier 48 for detecting photons emitted by the scintillation crystal 46. Detector electronics 73 containing a high voltage power supply and a signal processor is connected to the photomultiplier. Other electronic components required for NGS measurements will be understood by those skilled in the art of, for example, NGS wireline tools.

Preferably, the scintillation crystal 46 has a length extending along the axis of the analysis body 40. A relatively large scintillation crystal is preferred for capturing as much of the gamma-ray signal from the sample in the tray as possible. The scintillation crystal 46 is formed of a solid inorganic luminescent material that generates photons of light in response to contact with gamma-rays. Such inorganic luminescent materials include, for example, without limitation, sodium iodide (NaI), cesium iodide (CsI), and bismuth germanate (Bi4Ge3O12). Sodium iodide is a particularly preferred solid inorganic luminescent material for use in the in-line gamma-ray detection apparatus 10 of the present invention because relatively large sodium iodide crystals may be formed easily and economically.

The inorganic luminescent material may include one or more activators to enhance emission of photons by the scintillation crystal 46 that are within a range of wavelengths that are detectable by the photomultiplier 48. Such activators may be present as impurities in the scintillation crystal material and may be introduced to the crystal as a dopant. Thallium is a preferred activator for use in a sodium iodide or cesium iodide scintillation crystal utilized. A thallium-doped sodium iodide crystal is a preferred inorganic scintillation crystal material for use in the gamma-ray detector 44.

The photomultiplier 48 may be any conventional photomultiplier. As noted above, the photomultiplier 48 is optically coupled to the scintillation crystal 46, and may be physically coupled to the scintillation crystal 46 by locating an end of the scintillation crystal 46 in a receiving portion of the photomultiplier 48. Optical coupling grease, for example a silicon grease, may be applied at a contact interface between the end of the scintillation crystal 46 and the receiving portion of the photomultiplier 48 to reduce the loss of scintillation photons by preventing reflection of the photons at the contact interface. The photomultiplier 48 generates an electrical signal from detected photons of light emitted by the scintillation crystal 46 that is proportional to the gamma-ray energy absorbed in the scintillation crystal 46. The electrical signal produced by the photomultiplier 48 may be used to generate a gamma-ray spectrum for analysis.

The base 52 of the analysis body 40 houses the scintillation crystal 46. Preferably, the sample accumulation tray 42 covers the base 52 in such a way as to reduce penetration of fluids associated with the drill cuttings or weather into the base 52. In a preferred embodiment, the sample accumulation tray 42 is attached to the base 52 with a hinge 71, thereby allowing the opposing end of the sample accumulation tray 42 to move in response to weight for communicating with a weight sensor 66. This and other connections that allow for movement of the sample accumulation tray 42 in response to weight allows the weight sensor 66 to quantify the weight in the sample accumulation tray 42.

In one embodiment, the gamma ray detector 44 is coupled to the electronics enclosure 72 which encloses detector photomultiplier 48, detector electronics and any other electronic components required to operate the analysis body including communication module, weight sensor electronics, control module of an actuator 64 that moves wiper 58, power supplies, electric batteries supplying power, and the like. To reduce hazardous and potentially explosive interaction of hydrocarbon vapors present at the drilling rig with electronic components, the electronic enclosure 72 is sealed, preferably hermetically, and is filled with an electrically insulating fluid (not shown). An example of a suitable electrically insulating fluid is FLUORINERT™ available from 3M. Preferably, electronic components within the electronic enclosure 72 are connected with insulated, more preferably hermetically isolated, electrical connectors 74.

In another embodiment, the electronic enclosure 72 is coupled, preferably hermetically, to the gamma ray detector 44 and is filled with a gas at a pressure slightly higher than atmospheric pressure. Such positive pressure difference inside of electronics enclosure 72 reduces penetration of any hydrocarbon vapors in the environment surrounding the analysis body 40, thereby reducing the chance of explosion. This positive pressure difference can be maintained using an external gas reservoir (not shown) equipped with pressure control equipment and connected to electronics enclosure 72 by a gas line (not shown) as will be understood by those skilled in the art of electronics equipment isolation from hazardous and explosive substances.

The base 52 has a gamma ray detector shield 68. The detector shield 68 surrounds the gamma-ray detector 44 in such a way as to maximize suppression of gamma-ray signals emitted by any gamma-ray sources in the area of deployment, other than cuttings samples in the sample accumulation tray 42. The detector shield 68 is formed of a material having a high efficiency of gamma-ray absorption effective to reduce detection of any gamma-ray radiation beyond the gamma-rays emitted from the desired sample. In one embodiment, the material is lead. Alternatively, the detector shield 68 can be made of tungsten, W—Cu alloy, mercury or any other high-density material as it can be understood by those skilled in the art of radiation shielding design.

As shown in the embodiments depicted in FIGS. 3-7, the sample accumulation tray 42 has a shape for holding a pre-determined sample size. Material falling from the conveyor falls into the sample accumulation tray 42 so that the gamma-ray detector 44 can detect gamma-ray emissions from the material. The sample accumulation tray 42 allows the sample to be concentrated to produce a more meaningful result. After the gamma-rays are detected, the accumulated material is removed from the sample accumulation tray 42. Information about the gamma-ray spectra provided by the gamma-ray detector 44 is communicated to a control unit (not shown) that is remote from the distal end of the conveyor 150. Information is preferably communicated via a communication module (not shown) located inside the electronics enclosure 72.

Preferably, the weight of the material in the sample accumulation tray 42 is measured, for example, with a weight sensor 66. More preferably, accumulated material is removed from the sample accumulation tray 42 once the measured weight reaches a predetermined weight of material. Alternatively, accumulated material is removed from the sample accumulation tray 42 once a substantially constant weight of material is measured. It has been observed that the material falling from the conveyor 150 will fall onto any material in the sample accumulation tray 42 until a substantially steady state is reached where any new material rolls off the material already in place or displaces some material previously accumulated in the sample accumulation tray 42. As a further alternative, accumulated material is removed at a desired time interval or by external command.

When a desired sample size is accumulated in the sample accumulation tray 42, which is detected by the weight sensor 66, the sample is removed from the sample accumulation tray 42 by a sample removal device. A preferred device for removal is a wiper 58. In a preferred embodiment illustrated in FIGS. 3-7, the wiper 58 is carried by a yoke 62 that is adapted to travel along an actuator 64 along the length of the analysis body 40. The wiper 58 is preferably formed of a flexibly resilient material to conform with the shape of the sample accumulation tray 42, while being rigid enough to sweep or push the sample out of the sample accumulation tray 42. Preferably, the wiper 58 is formed of rubber, plastic or a combination thereof.

In a preferred embodiment illustrated in FIGS. 3-7, the actuator 64 is a pneumatic cylinder equipped with the two end switches and a valve controlled by the control module (not shown) located inside of the electronics enclosure 72 and connected to the external source of the pressurized gas (not shown) required for the actuator movement as it could be understood by those skilled in the art of pneumatic motion system design. The wiper 58 is connected by the yoke 62 to the actuator 64 and as a result the actuator 64 moves the wiper 58 along the length of the analysis body 40 from one side of the sample accumulation tray 42.

In the embodiment of FIGS. 3-7, the sample accumulation tray 42 has two free ends to allow movement of the wiper 58 along the length of the sample accumulation tray 42. Preferably, the yoke 62 allows the wiper 58 to travel past the free ends of the sample accumulation tray 42, thereby reducing any inadvertent trapping of sample between the wiper 58 and the sample accumulation tray 42.

The desired sample size is preferably measured with weight sensor 66, for example, as shown in FIG. 6. Other locations and types of weight sensors 66 may be used within the scope of the present invention.

In order to provide a quantitative analysis of the elements detected by the gamma-ray detector 44, weight information of the sample is also communicated via the communication module located inside the electronics enclosure 72 to the control unit so that the concentration of elements detected by the gamma-ray detector 44 can be quantified on a weight basis.

Preferably, the yoke 62 is actuated to move along the actuator 64 when the material in the sample accumulation tray 42 reaches a predetermined weight that is sensed by the weight sensor 66. In another preferred embodiment, the yoke 62 is actuated when the weight sensor 66 detects that the weight of material in the sample accumulation tray 42 is substantially constant. Alternatively, the yoke 62 may be actuated by a time interval or by external command sent to the apparatus through the communication line.

As an alternative to a wiper 58, a sample can be removed from the sample accumulation tray 42 by rotating the sample accumulation tray 42, pressure-washing, or other means within the scope of the present invention.

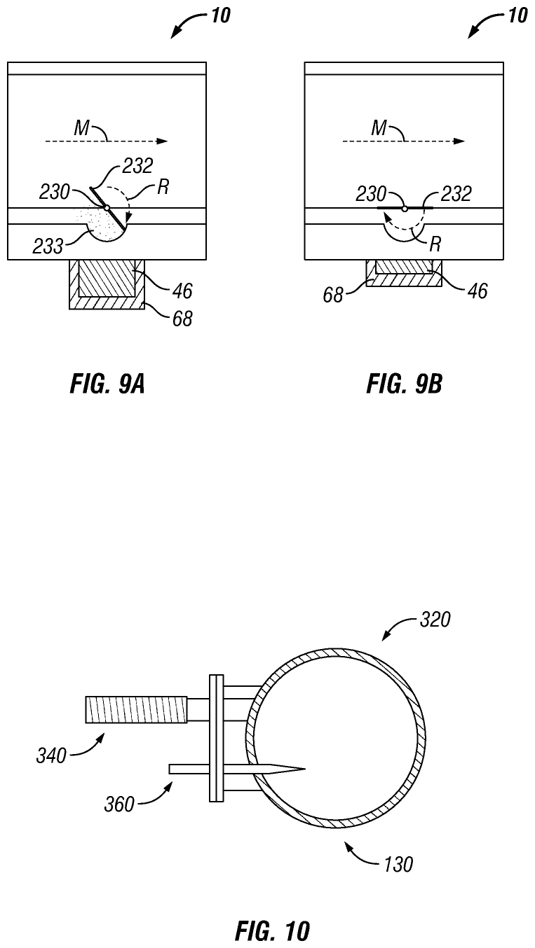

Another embodiment of the gamma-ray detection apparatus 10 for the system embodiment of FIG. 2 is depicted in FIGS. 8, 9A and 9B. The pipe insert 210 has a trap 220 cut in a base of the pipe insert 210. A rotating trap lid 230 is rotatably mounted on rotating pin 232. As illustrated more clearly in FIGS. 9A and 9B, the trap lid 230 is rotated about rotating pin 232 to swing the trap lid 230 into the flow of material, depicted by arrow M.

In this orientation, the trap lid 230 is rotated in a clockwise direction, depicted by arrow R. When the trap lid 230 is in the flow M, solids accumulate in the trap 220 (see FIG. 9A). When the trap lid 230 is out of the flow M, the material flow removes any solids concentrated in the trap 220 (see FIG. 9B).

A gamma-ray detector 44 capable of measuring spectrum of the detected gamma-ray signal preferably comprises a gamma-ray detecting scintillation crystal 46, which is preferably optically coupled to a photomultiplier (not shown) for detecting photons emitted by the scintillation crystal 46. The gamma-ray detector 44 is surrounded by the gamma-ray detector shield 68 made of the dense material, preferably lead. The detector shield 68 absorbs gamma-rays emitted by any gamma-ray sources present outside of the pipe insert 210, such as soil etc. The gamma-ray detector shield 68 reduces any contribution of gamma-ray radiation beyond the gamma-rays emitted from the desired sample.

Referring now to FIGS. 9A and 9B, in operation, the trap lid 230 is rotated into the flow of material, M, to concentrate solids 233 in the material. Gamma ray detector 44 acquires spectrum of the gamma-ray signal every several seconds. The amount of solid concentrated in the trap 220 is estimated by calculation and/or derived from measurements. Calculations may be based on known mud flow rate, rate of the penetration, trap shape or volume and other parameters. Alternatively, or in combination, measurements of concentrated solids volume using an ultra-sound sensor (not shown), measurements of concentrated solids weight using a weight sensor (not shown), and/or measurements by gamma ray densitometer (not shown), may be used. The information about the amount of concentrated solids in the trap is used to normalize gamma-ray spectra acquired by the gamma-ray detector 44 to convert the normalized spectra into concentration of U, K and Th in the solids. Preferably, the normalized gamma-ray spectra are corrected for any background source of gamma-rays. When the amount of concentered solids in the trap 220 reaches some threshold level or after a defined time interval the trap lid 230 is rotated (see FIG. 9B) to allow the material flow M to remove the concentrated solids 233 from the trap 220. After the trap 220 is cleaned by the material flow, for example, based on sensor readings or after a predefined time interval, the trap lid 230 is rotated into the material flow M to again concentrate solids.

Another component of the in-line mud logging system 100 is a hydrocarbon analysis device for in-line measuring hydrocarbon content in the material transported from the well 110. The hydrocarbon analysis device may be a gas-in-mud device, an oil-in-mud device or a combination thereof.

Gas-in mud devices are known to those skilled in the art and may include catalytic combustion or hot-wire detectors, thermal conductivity detectors, flame ionization detectors, gas chromatographs, infrared analyzers, mass spectrometry and combinations thereof. The devices quantify the concentration of hydrocarbons in mud gas. In particular, the concentration of aromatic compounds, such as benzene and toluene, can be quantified. By measuring the concentration of aromatic compounds, in the mud gas, information about the amount of oil in the formation can be determined.

The gas-in-mud devices may include a gas trap, and preferably, for example, a so-called constant volume trap (CVT). A CVT allows the material flow from the well through an extraction part of the CVT at a constant flow rate, thereby allowing measurements without being dependent on material flow rate through the possum belly 140 and potential complications due to blockage from oil accumulated in the possum belly 140.

An embodiment of an oil-in-mud device is depicted in FIG. 10. In this embodiment, a hydrocarbon analysis device 320 comprises an optical probe, for example a fluorescence probe, 340 for counting oil droplets in the material from the well 110. Aromatic compounds present in oil typically have strong fluorescence. As a result, the fluorescence probe 340 allows quantification of the amount of oil present in the drilling fluid by assessing the intensity of the fluorescent signal, which is proportional to the concentration of the aromatics compounds present in the drilling fluid. In the case of water based drilling fluid, the oil from the formation is suspended in the mud in the form of droplets. In this case, the fluorescence probe 340 quantifies oil droplets present in the drilling fluid and flowing past its tip in a given time interval, which in turn can be converted into the information about oil concentration.

In a preferred embodiment, the hydrocarbon analysis device 320 also comprises a sensor 360 for measuring conductivity, temperature or a combination thereof. In some instances, the information provided by the sensor 360 could decrease the uncertainty in the oil concentration determined from the data acquired by the optical probe.

The hydrocarbon analysis device 320 is disposed in the possum belly 140 or at another location of the in-line mud logging system 100 of the present invention suitable for in-line measurement of hydrocarbon content. Two suitable locations are shown in FIGS. 1 and 2, but the locations are not limited to the respective embodiments therein. For example, the embodiment of the in-line mud logging system 100 of FIG. 1 could have the hydrocarbon analysis device 320 in the location of FIG. 2, and vice versa.

In the embodiment of FIG. 1, the hydrocarbon analysis device 320 is disposed in communication with the mud pipeline 130 in a location between the well 110 and the possum belly 140. The hydrocarbon analysis device 320 may be placed directly in the mud pipeline 130 or in a by-pass line (not shown) parallel to the main flow of the mud pipeline 130.

In the embodiment of FIG. 2, the hydrocarbon analysis device 320 is disposed in communication with the possum belly 140.

Information about the gamma-ray spectra acquired by the gamma-ray detection apparatus 10 of any of the embodiments described herein is communicated to a control unit (not shown). Given the typical rate of drill cuttings produced and transported in a typical drilling operation, the sample size in the gamma-ray detection apparatus 10 may be reached every 20 to 30 seconds. Gamma-ray spectra can then be measured every several seconds to ensure a more accurate weight normalization of the resulting spectrum accumulated for each sample.

The control unit may comprise a database, processor, CPU, and/or any other computing device capable of receiving, processing, and/or storing data from the hydrocarbon analysis device 320 and the gamma-ray detection apparatus 10. The control unit may be structured and arranged to process information from the hydrocarbon analysis device 320 and the gamma-ray detection apparatus 10 to provide output data directed to the concentration of potassium, uranium, and/or thorium in the drill cutting sample and to the concentration of the toluene in the mud gas or oil in the drilling fluid.

Preferably, the gamma-ray spectra from the gamma-ray detection apparatus 10 are corrected for any background source of gamma-rays. A background spectrum is preferably measured in situ when there is no drilling and, as a result, no drill cuttings are produced. Preferably, the gamma-ray spectra are normalized by the control unit for a sample weight measured during the detection time.

Also, after normalization, an average gamma-ray spectrum may be calculated for gamma-ray spectra acquired between consecutive samples. The average gamma-ray spectrum corresponds to the gamma-ray spectrum of the formation averaged over the depth interval of interest. The decomposition of the average gamma-ray spectrum into K, U and Th contributions provides information about averaged concentrations of K, U and Th for the formation across the depth interval of interest.

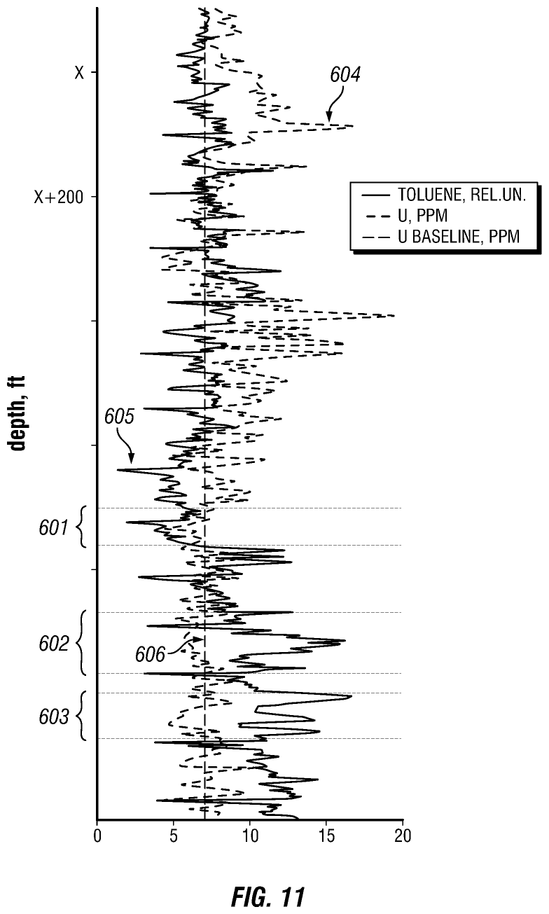

FIG. 11 is an example of sub-surface data that can be collected using one of the embodiments of the in-line mud logging system 100 of the present invention in the case of a formation containing shale and silt intervals. FIG. 11 graphically shows U concentration in the solids derived from gamma-ray spectra 604 and the concentration of toluene 605 in mud gas measured by mass spectrometer.

As discussed above, toluene is an aromatic hydrocarbon compound present in the oil that is dissolvable in water. So, the concentration of toluene in a drilling fluid is proportional to the amount of oil encountered by the drilling fluid when the drill bit penetrates particular formation intervals. Therefore, toluene in the mud gas that is detected by the hydrocarbon analysis device can be considered as a proxy for the oil present in the formation at corresponding depth.

Data presented in FIG. 11 allow for the identification of three tight silt intervals. U concentration in the formation in the case of intervals 601, 602 and 603 is below the average value marked by the vertical dashed line 606 indicating the presence of tight silt. Intervals 602 and 603 show spikes of toluene concentration, indicating the introduction of higher than average volumes of oil from the formation into drilling fluid. Only spikes that extend significantly above a background level of toluene concentration in mud gas would be taken into consideration. Indeed, in the case of this well no special cleaning of circulating water-based mud was employed. As a result, average concentration of toluene in water-based mud used to drill the well had been gradually increasing during penetration through a producible oil bearing formation resulting in gradual increase of the background toluene signal in the mass spectroscopy measurement. So, in the case of intervals 602 and 603, low U concentration in cuttings and high toluene concentration in the drilling mud indicate that these two intervals are tight silts charged with producible oil which could be targeted with lateral wells for the production.

The in-line mud logging system of the present invention may be used, for example, according to the method described in co-pending application entitled “In-Line Mud Logging Method” filed in the USPTO on the same day as the present application as a provisional application, the entirety of which is incorporated by reference herein.

While preferred embodiments of the present invention have been described, it should be understood that various changes, adaptations and modifications can be made therein within the scope of the invention(s) as claimed below.

1. An in-line mud logging system for real-time or near real-time analysis of material selected from drilling fluid, drill cuttings, mud gas and combinations thereof from a well, the material, having at least a solids content and a liquid content, the material transported from the well to a mud circulation system, the mud circulation system comprising a mud pipeline, a conveyor and a possum belly, the conveyor having a proximate end in material communication with the possum belly and a distal end from which the material falls from the conveyor, the in-line mud logging system comprising:
a hydrocarbon analysis device for in-line measuring hydrocarbon content in the material;
an in-line gamma-ray detection apparatus, the in-line gamma-ray detection apparatus disposed in the path of material transported from the well; and
a control unit for real-time or near real-time receiving and correlating signals from the hydrocarbon analysis device and the in-line gamma-ray detection apparatus.

2. The system of claim 1. wherein the in-line gamma-ray detection apparatus comprises:
a concentrator adapted to concentrate a portion of the solids content from the material;
a gamma-ray detector for acquiring spectra from the portion of the solids content; and
a sample removal device for removing the concentrated solids content from the concentrator.

3. The in-line-mud logging system of claim 2. wherein llte in-line gamma-ray detection apparatus further comprises a weight sensor, preferably wherein the weight sensor cooperates with the sample removal device.

4. The system of claim 2. wherein the in-line gamma-ray detection apparatus is disposed at the distal end of the conveyor.

5. The system of claim 2, wherein die in-line gamma-ray detection apparatus is disposed in the pipeline.

6. The system of claim 1, wherein the hydrocarbon analysis device is an oil-in-mud device, preferably wherein the hydrocarbon analysis device comprises a fluorescent probe adapted to count oil droplets in the drilling fluid, preferably wherein the hydrocarbon analysis device is selected from the group consisting of gas-in-mud devices, oil-in-mud devices, and combinations thereof, and preferably wherein the hydrocarbon analysis device further comprises a sensor selected from the group consisting of a conductivity sensor, a temperature sensor and combinations thereof.

7. The system of claim 1, wherein the material further comprises a gas content.

8. The system of claim 1, wherein the hydrocarbon analysis device is disposed in a mud pipeline between the well and the possum belly.

9. The system of claim 1. wherein the hydrocarbon analysis device is disposed in direct communication with the possum belly.

10. The system of claim 1, wherein the conveyor is a selected from the group consisting of a shaker conveyor, a vibratory conveyor, and combinations thereof.
